(19) United States
(12) Patent Application Publication
(10) Pub. No.: US 2023/0310068 A1
(43) Pub. Date: Oct. 5, 2023

(54) DUAL FOCAL AND LINEAR PULSE FIELD ABLATION (PFA) CATHETER

(71) Applicant: Medtronic, Inc., Minneapolis, MN (US)

(72) Inventors: Mark T. Stewart, Lino Lakes, MN (US); Brian T. Howard, Minneapolis, MN (US)

(21) Appl. No.: 18/183,527

(22) Filed: Mar. 14, 2023

Related U.S. Application Data
(60) Provisional application No. 63/326,482, filed on Apr. 1, 2022.

(51) Int. Cl. A61B 18/14

(52) U.S. Cl. CPC A61B 18/1492; A61B 2018/1407; A61B 2018/1467; A61B 2018/1435; A61B 2018/00577

(57) ABSTRACT

An example catheter for performing pulsed field ablation (PFA) includes: an elongated structure defining a longitudinal axis; a plurality of electrodes carried on a distal portion of the elongated structure, the plurality of electrodes comprising: a tip electrode positioned at a distal tip of the elongated structure; a tip ring electrode adjacent to the tip electrode; a pair of ring electrodes; and one or more additional electrodes, wherein the pair of ring electrodes is disposed longitudinally along the elongated structure between the tip ring electrode and the one or more additional electrodes.

This application claims the benefit of U.S. Provisional Patent Application 63/326,482, which was filed on Apr. 1, 2022, the entire content of which is incorporated herein by reference.

### TECHNICAL FIELD

The present technology is related to ablation catheters. In particular, various examples of the present technology are related to ablation catheters for performing pulse field ablation (PFA).

### BACKGROUND

Tissue ablation is a medical procedure commonly used to treat conditions such as cardiac arrhythmia, which includes atrial fibrillation. For treating cardiac arrhythmia, ablation can be performed to modify tissue, such as to stop aberrant electrical propagation and/or disrupt aberrant electrical conduction through cardiac tissue. Although thermal ablation techniques are frequently used, such as cryoablation and radiofrequency (RF) ablation, non-thermal techniques such as pulsed field ablation (PFA) may also be used.

Pulsed field ablation involves the application of short pulsed electric fields (PEF), which may reversibly or irreversibly destabilize cell membranes through electropermeabilization, but generally do not affect the structural integrity of the tissue components, including the acellular cardiac extracellular matrix. The nature of PFA allows for very brief periods of therapeutic energy delivery, on the order of tens of milliseconds in duration. Further, PFA may not cause collateral damage to non-targeted tissue as frequently or severely as thermal ablation techniques.

### SUMMARY

The present technology is directed to devices, systems, and methods for performing pulsed field ablation (PFA) in multiple modes using a single catheter. PFA may be performed in multiple modes, including a linear mode and a focal mode. Linear mode PFA may result in a field geometry that is relatively even linearly along an active portion of a catheter (e.g., cathodes and anodes may be alternating along the catheter). On the other hand, focal mode PFA may result in a field geometry that is focused at a tip of a catheter (e.g., a tip of the catheter may be a cathode and while other portions of the catheter may be anodes, or vice versa). In general, linear mode PFA may generate a lesion that is longer than it is wide, whereas focal mode PFA may generate a lesion that is wider than it is long. In some examples, a practitioner who desires to use both linear mode PFA and focal mode PFA may have to physically switch between different catheters. For instance, to switch from linear mode PFA to focal mode PFA, the surgeon may have to remove a linear mode catheter and insert a focal mode catheter. Having to physically switch catheters when using different PFA modes may be undesirable.

In accordance with one or more aspects of this disclosure, a single catheter may be configured to perform PFA in both a linear mode and a focal mode. For instance, the catheter may include multiple electrodes that are independently controllable and in sufficient quantity to perform both linear and focal modes. To operate the catheter in the focal mode, a controller may output energy to electrodes of the catheter to cause the electrodes to generate a field with a geometry focused at a tip of the catheter. For instance, the controller may cause one or more electrodes positioned proximal to a tip of the catheter to operate as a cathode and cause a plurality of electrodes positioned more distal from the tip to operate as anodes. To operate the catheter in the linear mode, the controller may output energy to the electrodes of the catheter to cause the electrodes to generate a field with a geometry that is relatively even linearly along an active portion of a catheter. For instance, the controller may cause the electrodes to form alternating cathodes and anodes.

While a catheter capable of performing both linear and focal PFA may be more complex and/or more expensive to manufacture than a catheter capable of performing only one of linear and focal PFA, the catheter of this disclosure may provide several advantages. For instance, the catheter of this disclosure may enable a surgeon to switch between linear and focal PFA without removal and re-insertion of a catheter.

In one example, a catheter for performing pulse field ablation (PFA) includes an elongated structure defining a longitudinal axis; a plurality of electrodes carried on a distal portion of the elongated structure, the plurality of electrodes comprising: a tip electrode positioned at a distal tip of the elongated structure; a tip ring electrode adjacent to the tip electrode; a pair of ring electrodes; and one or more additional electrodes, wherein the pair of ring electrodes is disposed longitudinally along the elongated structure between the tip ring electrode and the one or more additional electrodes.

In another example, a method for performing pulse field ablation (PFA) includes determining, by a controller connected to a particular catheter and at a first time, to perform PFA using a linear PFA mode; responsive to determining to use the linear PFA mode, outputting, by the controller and to electrodes of the particular catheter, energy to cause the electrodes to generate a field with a geometry that is linear along an active portion of the particular catheter; determining, by the controller and at a second time, to perform PFA using a focal PFA mode; and responsive to determining to use the focal PFA mode, outputting, by the controller and to the electrodes of the particular catheter, energy to cause the electrodes to generate a field with a geometry that is focused at a tip of the particular catheter.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example system for delivering pulsed field ablation (PFA), in accordance with one or more aspects of this disclosure.

FIGS. 2A and 2B are conceptual diagrams illustrating example linear and focal operation of a catheter, in accordance with one or more aspects of this disclosure.

FIGS. 2C and 2D are conceptual diagrams illustrating example fields resulting from linear and focal operation of a catheter, in accordance with one or more aspects of this disclosure.

FIGS. 3A and 3B are conceptual diagrams illustrating example linear and focal operation of a catheter, in accordance with one or more aspects of this disclosure.

FIGS. 3C and 3D are conceptual diagrams illustrating example fields resulting from linear and focal operation of a catheter, in accordance with one or more aspects of this disclosure.

FIG. 4 is a conceptual diagram illustrating an example focal operation of a catheter, in accordance with one or more aspects of this disclosure.

FIG. 5 is a block diagram illustrating an example controller of a multi-mode PFA system, in accordance with one or more aspects of this disclosure.

FIG. 6 is a flowchart illustrating an example technique for using a single catheter to perform both linear and focal PFA, in accordance with one or more techniques of this disclosure.

### DETAILED DESCRIPTION

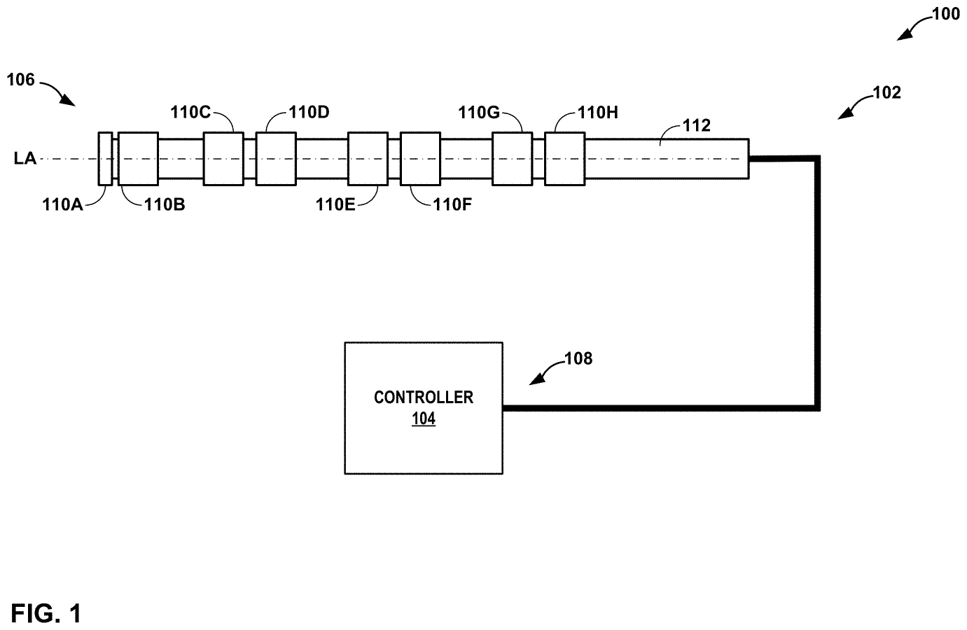

FIG. 1 is a conceptual diagram illustrating an example system 100 for delivering pulsed field ablation (PFA) that includes a catheter 102 and a controller 104. In general, to deliver PFA, a practitioner (e.g., cardiologist, surgeon, etc.) may insert catheter 102 into a patient and cause controller 104 to deliver, via catheter 102, electroporation energy (e.g., pulsed field ablation energy). Electroporation may be a phenomenon causing cell membranes to become “leaky” (that is, permeable for molecules for which the cell membrane may otherwise be impermeable or semipermeable). Electroporation, which may also be referred to as electropermeabilization, pulsed electric field treatment, non-thermal irreversible electroporation, irreversible electroporation, high frequency irreversible electroporation, nanosecond electroporation, or nanoelectroporation, may involve the application of high-amplitude pulses to cause physiological modification (i.e., permeabilization) of the cells of the tissue to which the energy is applied. These pulses may be short (for example, nanosecond, microsecond, or millisecond pulse width) in order to allow the application of high voltage, high current (for example, 20 or more amps) without long duration(s) of electrical current flow that may otherwise cause significant tissue heating and muscle stimulation. The pulsed electric energy may induce the formation of microscopic defects that result in hyperpermeabilization of the cell membrane. Depending on the characteristics of the electrical pulses, an electroporated cell can survive electroporation, referred to as “reversible electroporation,” or die, referred to as “irreversible electroporation” (IRE). Reversible electroporation may be used to transfer agents, including genetic material and other large or small molecules, into targeted cells for various purposes, including the alteration of the action potentials of cardiac myocytes.

Catheter 102 may include elongated structure 112 carrying a plurality of electrodes 110A-110H (collectively, “electrodes 110”). Catheter 102 may generally include features that enable insertion of catheter 102 into a patient and navigation of catheter 102 to a target treatment site. Elongated structure 112 may include a distal portion 106 and a proximal portion 108. Electrodes 110 may be generally positioned at distal portion 106, while proximal portion 108 may be connected to controller 104. Electrodes 110 may be of any suitable geometry. Example geometries of electrodes include, but are not necessarily limited to, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes). Electrodes 110 may be axially distributed along longitudinal axis LA of elongated structure 112.

Elongated structure 112 may include conductors configured to carry electrical signals between electrodes 110 and controller 104. In some examples, elongated structure 112 may include a separate conductor (e.g., a separate control lead) for each of electrodes 110. For instance, in the example of FIG. 1 where electrodes 110 includes eight electrodes, elongated structure 112 may include eight separate conductors. In this way, elongated structure may enable each electrode of electrodes 110 to be driven with a different signal. In other examples, multiple electrodes of electrodes 110 may share a common conductor. For instance, electrodes 110C and 110D may be connected to a same (e.g., a common) conductor. While such a common conductor arrangement may reduce flexibility (e.g., as electrodes connected to the common conductor may be driven with a same signal), such an arrangement may reduce manufacturing complexity and/or cost. In general, the conductors may be referred to as control leads.

As shown in FIG. 1, electrodes 110 may include a tip electrode (e.g., electrode 110A), which may be a ring electrode with a “cap” covering at least a portion of a tip of elongated structure 112. In some examples, the tip electrode may be chamfered or otherwise rounded (e.g., to enable easier passage of catheter 102 through anatomy of the patient). Electrodes 110 may include a tip ring electrode (e.g., electrode 110B) that is adjacent to the tip electrode. The tip ring electrode may be separated (axially along LA) from the tip electrode. Electrodes 110 may include one or more pairs of ring electrodes. A pair of ring electrodes may include two adjacently closely spaced electrodes of electrodes 110. For instance, in the example of FIG. 1, electrodes 110C and 110D may form a first pair of ring electrodes, electrodes 110E and 110F may form a second pair of ring electrodes, and electrodes 110G and 110H may form a third pair of ring electrodes. In general, the first pair of ring electrodes (i.e., electrodes 110C and 110D) may be accompanied by one or more additional electrodes. The one or more additional electrodes may include any combination of pairs of ring electrodes and coil electrodes (e.g., electrodes that include conductors that spiral around elongated structure 112). As such, electrodes 110 may include tip electrode 110A, tip ring electrode 110B (e.g., adjacent to tip electrode 110A), electrodes 110C and 110D (e.g., a pair of ring electrodes), and one or more additional electrodes (e.g., pair of ring electrodes 110E and 110F, and pair of ring electrodes 110G and 110H).

In the example of FIG. 1, electrodes 110 are illustrated as has having a larger diameter than elongated structure 112. In some examples, one or more of electrodes 110 may have a diameter that is approximately equal to a diameter of elongated structure 112. For instance, electrodes 110 may be recessed in elongated structure 112 such that the combination results in a relatively smooth outer surface.

Controller 104 may include an energy generator configured to provide electrical pulses to electrodes 110 to perform an electroporation procedure to cardiac tissue or other tissues within the patient's body, such as renal tissue, airway tissue, and organs or tissue within the cardiac space or the pericardial space. For instance, the energy generator may be configured and programmed to deliver pulsed, high-voltage electric fields appropriate for achieving desired pulsed, high-voltage ablation (referred to as “pulsed field ablation” or “pulsed electric field ablation”) and/or pulsed radiofrequency ablation. As a point of reference, the non-radiofrequency pulsed high-voltage ablation effects of the present disclosure are distinguishable from DC current ablation, as well as thermally-induced ablation attendant with conventional RF techniques. For example, the pulse trains delivered by the energy generator may be delivered at a frequency less than 30 kHz, and in an exemplary configuration, 1 kHz, which is a lower frequency than radiofrequency treatments. The pulsed-field energy in accordance with the present disclosure may be sufficient to induce cell death for purposes of completely blocking an aberrant conductive pathway along or through cardiac tissue, destroying the ability of the so-ablated cardiac tissue to propagate or conduct cardiac depolarization waveforms and associated electrical signals. Additionally or alternatively, the energy generator may be configured and programmed to deliver RF energy appropriate for achieving tissue ablation.

In accordance with one or more aspects of this disclosure, catheter 102 may be configured to selectably perform PFA using a linear mode or a focal mode. For instance, electrodes 110 of catheter 102 may include both electrodes configured to deliver PFA using linear mode and electrodes configured to deliver PFA using focal mode (there may be some or total overlap between the electrodes for the different modes). By enabling a single catheter to perform both linear and focal mode PFA, this disclosure provides several advantages. As one example, a practitioner may switch between linear and focal lesion formation without having to remove the catheter. As another example, supply management may be simplified (e.g., as a quantity of catheter types may be reduced).

During an ablation procedure, a practitioner may desire to switch between linear and focal lesion formation. To achieve such a switching, controller 104 may be configured to selectably operate catheter 102 in the linear mode and the focal mode. The practitioner may adjust a setting of controller 104 to be linear mode or focal mode. Controller 104 may operate catheter 102 in the selected mode. For instance, to operate catheter 102 in the focal mode, controller 104 may output energy to electrodes 110 to cause electrodes 110 to generate a field with a geometry focused at a tip of catheter 102 (e.g., focused at tip electrode 110A). Such a focused field geometry may result in lesions forming proximal to the tip of catheter 102. To operate catheter 1002 in the linear mode, controller 104 may output energy to electrodes 110 to cause electrodes 110 to generate a field with a geometry that is relatively even linearly along an active portion of catheter 102 (e.g., along a portion of catheter 102 on which electrodes 110 are positioned). Such a linear field geometry may result in lesions forming longitudinally along the active portion of catheter 102.

As discussed above, to operate catheter 102 in the focal mode, controller 104 may output energy to electrodes 110 to cause electrodes 110 to generate a field with a geometry focused at a tip of catheter 102. For instance, controller 104 may cause one or more electrodes positioned proximal to a tip of catheter 102 (e.g., tip electrode 110A and tip ring electrode 110B) to operate as cathodes and cause a plurality of electrodes positioned more distal from the tip (e.g., ring electrodes 110C and 110D) to operate as anodes. In some examples, one or more of the cathodes and/or anodes may be formed of a single electrode. In some examples, one or more of the cathodes and/or anodes may be formed of multiple electrodes. For instance, electrodes of a pair of ring electrodes may be driven with a same signal such that the electrodes of the pair of ring electrodes act as a single cathode or anode.

In some examples, to facilitate acting as a single anode or cathode, electrodes of a pair of ring electrodes may be positioned closer to each other than to adjacent electrodes. For instance, a distance between electrodes of a first pair of ring electrodes (e.g., a distance along LA between electrodes 110C and 110D) may be less than a distance between a distal ring electrode of the pair of ring electrodes and the tip ring electrode (e.g., a distance along LA between electrode 110C and electrode 110B).

Distances between adjacent pairs of ring electrodes may be approximately equal. For instance, a distance between the first pair of ring electrodes (e.g., ring electrodes 110C and 110D) and the second pair of ring electrodes (e.g., ring electrodes 110E and 110F) may be approximately equal to a distance between the second pair of ring electrodes and a third pair of ring electrodes (e.g., ring electrodes 110G and 110H). As such, in some examples, pairs of ring electrodes of electrodes 110 may be considered to be equally spaced along LA of catheter 102.

Although not shown, system 100 may include one or more sensors to monitor the operating parameters through the medical system 100, such as temperature, delivered voltage, or the like, and for measuring and monitoring one or more tissue characteristics, such as EGM waveforms, monophasic action potentials, tissue impedance, or the like, in addition to monitoring, recording, or otherwise conveying measurements or conditions within the energy delivery device or other component of system 100 or the ambient environment at the distal portion of the energy delivery device. The sensor(s) may be in communication with controller 104 for initiating or triggering one or more alerts or ablation energy delivery modifications during operation of the energy delivery device.

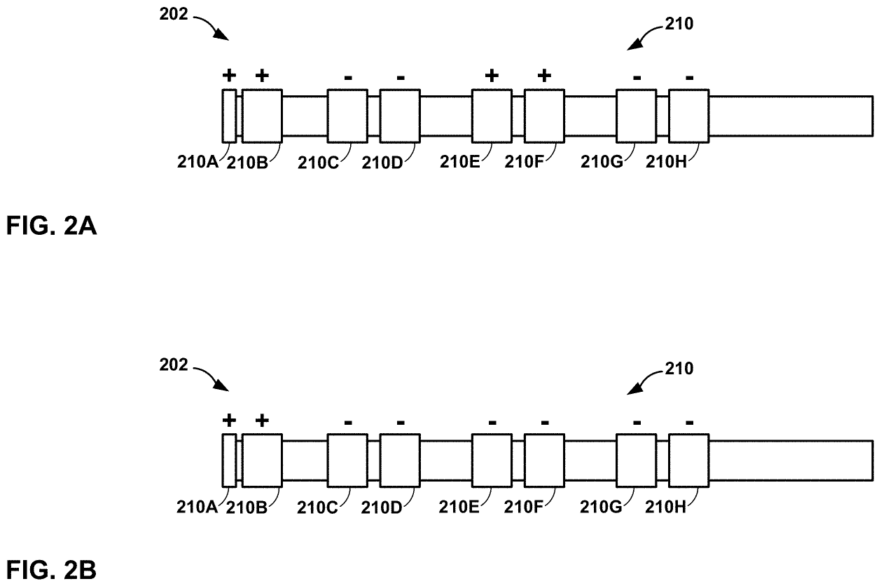

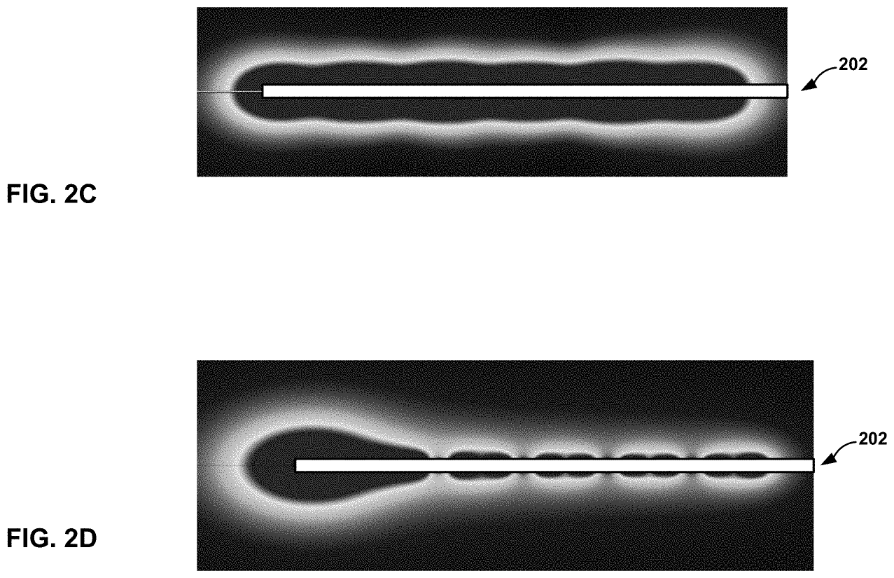

FIGS. 2A and 2B are conceptual diagrams illustrating example linear and focal operation of a catheter, in accordance with one or more aspects of this disclosure. FIGS. 2C and 2D are conceptual diagrams illustrating example fields resulting from linear and focal operation of a catheter, in accordance with one or more aspects of this disclosure. Catheter 202 of FIGS. 2A and 2B may be an example of catheter 102 of FIG. 1.

FIG. 2A illustrates an example of how a controller, such as controller 104, may operate catheter 202 in the linear PFA mode. As shown in FIG. 2A, to operate catheter 302 in the linear PFA mode, the controller may drive electrode 210A (e.g., a tip electrode) and electrode 210B (e.g., a tip ring electrode) at a first polarity (e.g., as a cathode in the example of FIG. 2A). The controller may drive electrodes 210C and 210D (e.g., a first pair of ring electrodes) at a second polarity that is different than the first polarity (e.g., as an anode in the example of FIG. 2A). The controller may drive electrodes 210E and 210F (e.g., a second pair of ring electrodes) at the first polarity, and drive electrodes 210G and 210H (e.g., a third pair of ring electrodes) at the second polarity. By driving electrodes 210 (pairs of electrodes 210) at alternating polarities, the controller may achieve a substantially linear field axially along catheter 202 as shown in FIG. 2C. In general, driving an electrode may include delivering energy to the electrode. For instance, to drive electrodes 210 at alternating polarities, controller 104 may deliver energy to pairs of electrodes 210 at alternating polarities.

In general, electrodes of the pairs of ring electrodes may be closer together than electrodes of adjacent pairs. For instance, a distance between electrode 210C and electrode 210D may be less than a distance between electrode 210D and electrode 210E.

FIG. 2B illustrates an example of how a controller, such as controller 104, may operate catheter 202 in the focal PFA mode. As shown in FIG. 2B, to operate catheter 302 in the focal PFA mode, the controller may drive electrode 210A (e.g., a tip electrode) and electrode 210B (e.g., a tip ring electrode) at a first polarity (e.g., as a cathode in the example of FIG. 2A). The controller may drive electrodes 210C and 210D (e.g., a first pair of ring electrodes) at a second polarity that is different than the first polarity (e.g., as an anode in the example of FIG. 2A). The controller may drive electrodes 210E and 210F (e.g., a second pair of ring electrodes) at the second polarity, and drive electrodes 210G and 210H (e.g., a third pair of ring electrodes) at the second polarity. By driving electrodes of electrodes 210 near the tip at the first polarity and driving other electrodes of electrodes 210 at the second polarity, the controller may achieve a substantially focuses field at the tip of catheter 202 as shown in FIG. 2D.

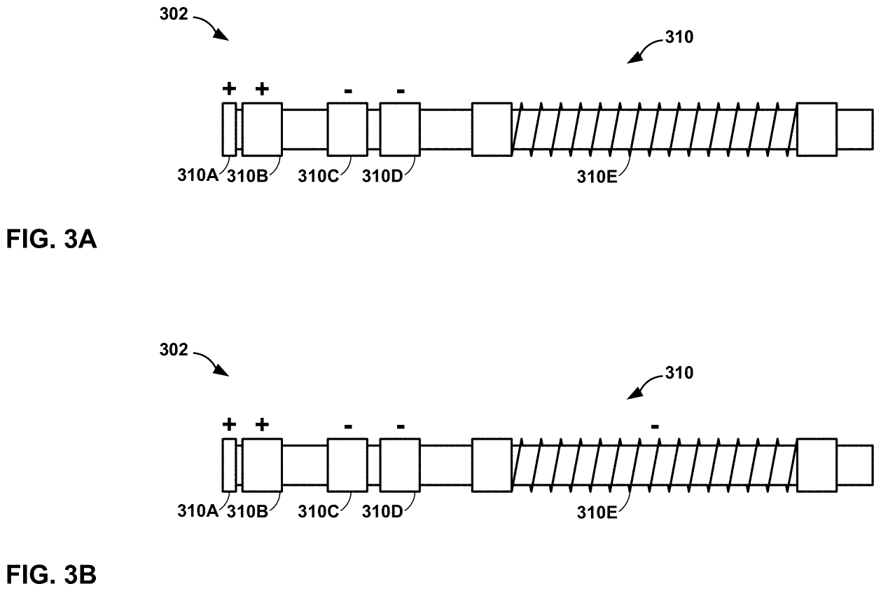

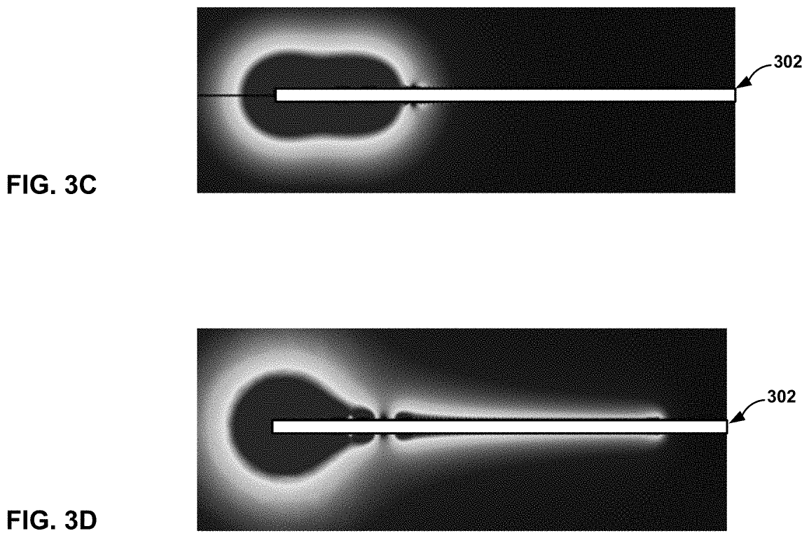

FIGS. 3A and 3B are conceptual diagrams illustrating example linear and focal operation of a catheter, in accordance with one or more aspects of this disclosure. FIGS. 3C and 3D are conceptual diagrams illustrating example fields resulting from linear and focal operation of a catheter, in accordance with one or more aspects of this disclosure. Catheter 302 of FIGS. 3A and 3B may be an example of catheter 102 of FIG. 1.

FIG. 3A illustrates an example of how a controller, such as controller 104, may operate catheter 302 in the linear PFA mode. As shown in FIG. 3A, to operate catheter 302 in the linear PFA mode, the controller may drive electrode 310A (e.g., a tip electrode) and electrode 310B (e.g., a tip ring electrode) at a first polarity (e.g., as a cathode in the example of FIG. 3A). The controller may drive electrodes 310C and 310D (e.g., a first pair of ring electrodes) at a second polarity that is different than the first polarity (e.g., as an anode in the example of FIG. 3A). In the linear mode, the controller may not drive electrode 310E (e.g., a coil electrode). For instance, the controller may let electrode 310E float. By driving electrodes 310 in this way, the controller may achieve a substantially linear field axially along catheter 302 as shown in FIG. 3C. As such, electrodes 310 may include tip electrode 310A, tip ring electrode 310B (e.g., adjacent to tip electrode 310A), electrodes 310C and 310D (e.g., a pair of ring electrodes), and one or more additional electrodes (e.g., coil electrode 310E).

As noted above, an electrode (e.g., electrode 310E) may be a coil electrode. A coil could take the form of a wound wire coil or alternatively, as a spiral laser cut tube which is flexible like a coil but has a larger surface area. The number of wraps may be adjusted to decrease or increase surface area at the ends or middle portion of the coil. In one example, the surface area at the distal end of the coil could be reduced (wider spaced coil wraps). In this way, aspects of this disclosure may limit the electric field distribution around that end region of the coil. In some examples, this may an example of a short linear mode.

FIG. 3B illustrates an example of how a controller, such as controller 104, may operate catheter 302 in the focal PFA mode. As shown in FIG. 3B, to operate catheter 302 in the focal PFA mode, the controller may drive electrode 210A (e.g., a tip electrode) and electrode 210B (e.g., a tip ring electrode) at a first polarity (e.g., as a cathode in the example of FIG. 2A). The controller may drive electrodes 210C and 210D (e.g., a first pair of ring electrodes) at a second polarity that is different than the first polarity (e.g., as an anode in the example of FIG. 2A). The controller may drive electrode 210E (e.g., the coil electrode) at the second polarity. By driving electrodes of electrodes 310 in this way, the controller may achieve a substantially focuses field at the tip of catheter 302 as shown in FIG. 3D.

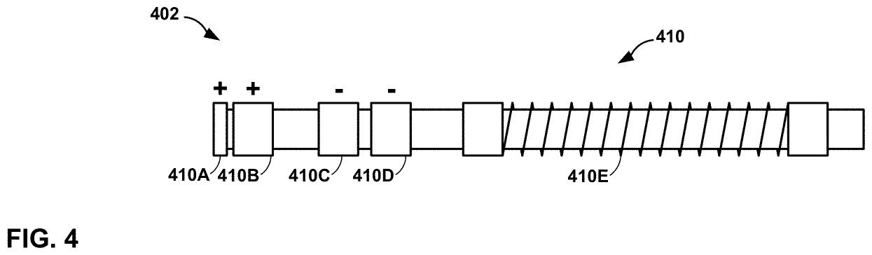

FIG. 4 is a conceptual diagram illustrating an example focal operation of a catheter, in accordance with one or more aspects of this disclosure. In particular, FIG. 4 illustrates an example of how a controller, such as controller 104, may operate catheter 402 in the linear PFA mode. As shown in FIG. 4, to operate catheter 402 in the linear PFA mode, the controller may drive electrode 410A (e.g., a tip electrode) and electrode 410B (e.g., a tip ring electrode) at a first polarity (e.g., as a cathode in the example of FIG. 4). The controller may let electrodes 310C and 310D (e.g., a first pair of ring electrodes) float and drive electrode 410E (e.g., a coil electrode) at a second polarity that is different than the first polarity (e.g., as an anode in the example of FIG. 4). By driving electrodes 410 in this way, the controller may produce a more focused focal mode with ablation occurring more closely to two tip electrodes 410A and 410B. In some examples, this may an example of a long linear mode.

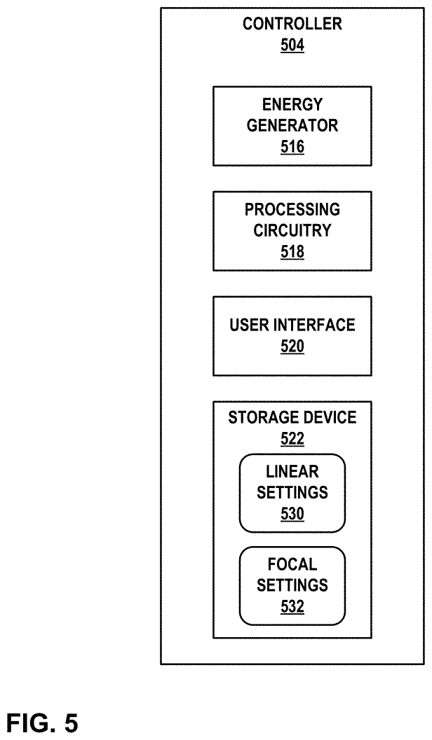

FIG. 5 is a block diagram illustrating an example controller of a multi-mode PFA system, in accordance with one or more aspects of this disclosure. Controller 504 of FIG. 5 may be an example of controller 104 of FIG. 1. As shown in FIG. 5, controller 504 may include energy generator 516, processing circuitry 518, user interface 520, and storage devices 522.

Energy generator 516 may be configured to provide electrical pulses to electrodes (e.g., electrodes 110 of FIG. 1) to perform an electroporation procedure to cardiac tissue or other tissues within the patient's body, such as renal tissue, airway tissue, and organs or tissue within the cardiac space or the pericardial space. For instance, energy generator 516 may be configured and programmed to deliver pulsed, high-voltage electric fields appropriate for achieving desired pulsed, high-voltage ablation (referred to as “pulsed field ablation” or “pulsed electric field ablation”) and/or pulsed radiofrequency ablation. While shown in the example of FIG. 5 as a single energy generator, energy generator 516 is not so limited. For instance, controller 504 may include multiple energy generators that are each capable of generating ablation signals in parallel.

Processing circuitry 518 may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 518 herein may be embodied as firmware, hardware, software or any combination thereof. Processing circuitry 518 controls energy generator 516 to generate signals according to various settings (e.g., linear settings 530 or focal settings 532). In some examples, processing circuitry 518 may execute other instructions stored in storage device 522 to perform PFA in accordance with linear settings 530 or focal settings 532.

Storage device 522 may be configured to store information within controller 504, respectively, during operation. Storage device 522 may include a computer-readable storage medium or computer-readable storage device. In some examples, storage device 522 includes one or more of a short-term memory or a long-term memory. Storage device 522 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 522 is used to store data indicative of instructions, e.g., for execution by processing circuitry 518, respectively. As discussed above, storage device 522 is configured to store linear settings 430 and focal settings 532.

User interface 520 may include a button or keypad, lights, a speaker for voice commands, a display, such as a liquid crystal (LCD), light-emitting diode (LED), or organic light-emitting diode (OLED). In some examples, the display includes a touch screen. User interface 520 may be configured to display any information related to the performance of PFA. User interface 520 may also receive user input (e.g., selection of linear or focal PFA mode) via user interface 520. The user input may be, for example, in the form of pressing a button on a keypad or selecting an icon from a touch screen.

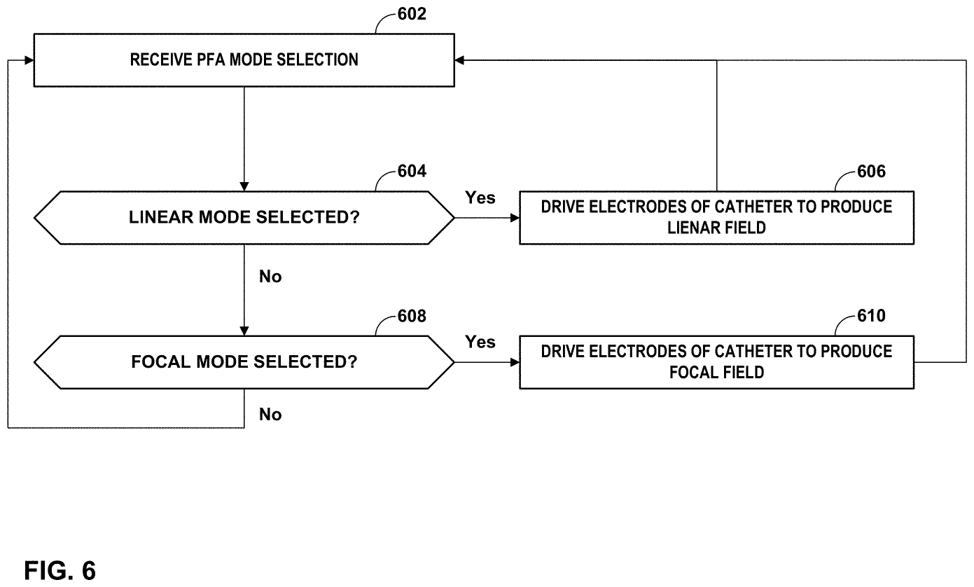

FIG. 6 is a flowchart illustrating an example technique for using a single catheter to perform both linear and focal PFA, in accordance with one or more techniques of this disclosure. The technique of FIG. 6 may be performed by a controller, such as controller 104 of FIG. 1 or controller 504 of FIG. 5.

Controller 504 may receive a PFA model selection (602). For instance, processing circuitry 518 of controller 504 may receive, via user input devices 520, a selection from a practitioner whether to operate in a focal mode or a linear mode.

Controller 504 may determine whether the linear mode is selected (604). Responsive to determining to use the linear PFA mode (e.g., “yes” branch of 604), controller 504 may drive electrodes of a catheter to produce a linear field (606). For instance, processing circuitry 514 may cause energy generator 516 to output, to electrodes of the catheter, energy to cause the electrodes to generate a field with a geometry that is linear along an active portion of the particular catheter (e.g., similar to the fields illustrated in FIGS. 2C and 3C).

Controller 504 may determine whether the focal mode is selected (608). Responsive to determining to use the focal PFA mode (e.g., “yes” branch of 608), controller 504 may drive electrodes of a catheter to produce a focal field (610). For instance, processing circuitry 514 may cause energy generator 516 to output, to electrodes of the catheter, energy to cause the electrodes to generate a field with a geometry that is focused at a tip of the particular catheter (e.g., similar to the fields illustrated in FIGS. 2D and 3D).

The following numbered examples may illustrate one or more aspects of the disclosure:

Example 1. A catheter for performing pulsed field ablation (PFA), the catheter comprising: an elongated structure defining a longitudinal axis; a plurality of electrodes carried on a distal portion of the elongated structure, the plurality of electrodes comprising: a tip electrode positioned at a distal tip of the elongated structure; a tip ring electrode adjacent to the tip electrode; a pair of ring electrodes; and one or more additional electrodes, wherein the pair of ring electrodes is disposed longitudinally along the elongated structure between the tip ring electrode and the one or more additional electrodes.

Example 2. The catheter of example 1, wherein a distance between electrodes of the pair of ring electrodes is less than a distance between a distal ring electrode of the pair of ring electrodes and the tip ring electrode.

Example 3. The catheter of example 1 or example 2, further comprising: a first common control lead connected to the tip electrode and the tip ring electrode.

Example 4. The catheter of any of examples 1-3, further comprising: a second common control lead connected to both electrodes of the pair of ring electrodes.

Example 5. The catheter of example 1 or example 2, further comprising: a separate control lead for each electrode of the plurality of electrodes.

Example 6. The catheter of any of examples 1-5, wherein the one or more additional electrodes comprises a coil electrode.

Example 7. The catheter of any of examples 1-6, wherein the one or more additional electrodes comprises one or more additional pairs of ring electrodes.

Example 8. The catheter of any of examples 1-5, wherein the one or more additional electrodes comprises: a coil electrode; and one or more additional pairs of ring electrodes disposed longitudinally along the elongated structure between the coil electrode and the pair of ring electrodes.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof. Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media. It should also be understood that when describing electrodes as being anodes or cathodes, this is not intended to imply that direct current is being delivered but in general, this disclosure uses such terms to denote that electrodes which are called anodes are connected to the opposite polarity of those called cathodes when delivering alternating current or most commonly, biphasic pulsed waveforms. Such biphasic waveforms may be delivered as a series of pulses (pulse train) that consists of a positive square wave pulse followed by a negative square wave pulse where such a pulse train may consist of tens or hundreds of such alternating polarity (biphasic) pulses.

What is claimed is:

1. A catheter for performing pulsed field ablation (PFA), the catheter comprising:
an elongated structure defining a longitudinal axis;
a plurality of electrodes carried on a distal portion of the elongated structure, the plurality of electrodes comprising:
a tip electrode positioned at a distal tip of the elongated structure;
a tip ring electrode adjacent to the tip electrode;
a pair of ring electrodes; and
one or more additional electrodes, wherein the pair of ring electrodes is disposed longitudinally along the elongated structure between the tip ring electrode and the one or more additional electrodes.

2. The catheter of claim 1, wherein a distance between electrodes of the pair of ring electrodes is less than a distance between a distal ring electrode of the pair of ring electrodes and the tip ring electrode.

3. The catheter of claim 1, further comprising:
a first common control lead connected to the tip electrode and the tip ring electrode.

4. The catheter of claim 1, further comprising:
a second common control lead connected to both electrodes of the pair of ring electrodes.

5. The catheter of claim 1, further comprising:
a separate control lead for each electrode of the plurality of electrodes.

6. The catheter of claim 1, wherein the one or more additional electrodes comprises a coil electrode.

7. The catheter of claim 1, wherein the one or more additional electrodes comprises one or more additional pairs of ring electrodes.

8. The catheter of claim 1, wherein the one or more additional electrodes comprises:
a coil electrode; and
one or more additional pairs of ring electrodes disposed longitudinally along the elongated structure between the coil electrode and the pair of ring electrodes.

9. A system comprising:
a catheter for performing pulsed field ablation (PFA), the catheter comprising:
an elongated structure defining a longitudinal axis;
a plurality of electrodes carried on a distal portion of the elongated structure, the plurality of electrodes comprising:
a tip electrode positioned at a distal tip of the elongated structure;
a tip ring electrode adjacent to the tip electrode;
a pair of ring electrodes; and
one or more additional electrodes, wherein the pair of ring electrodes is disposed longitudinally along the elongated structure between the tip ring electrode and the one or more additional electrodes.

10. The system of claim 9, further comprising:
a controller configured to control delivery of electrical stimulation via the catheter to perform PFA.

11. A catheter for performing pulsed field ablation (PFA), the catheter comprising:
an elongated structure defining a longitudinal axis;
a plurality of electrodes carried on a distal portion of the elongated structure, the plurality of electrodes comprising:
a tip electrode positioned at a distal tip of the elongated structure;
a tip ring electrode adjacent to the tip electrode;
a pair of ring electrodes; and
one or more additional electrodes, wherein the pair of ring electrodes is disposed longitudinally along the elongated structure between the tip ring electrode and the one or more additional electrodes;
a plurality of control leads extending through the elongated structure, the plurality of control leads including:
a first common control lead connected to both the tip electrode and the tip ring electrode; and
a second common control lead connected to both electrodes of the pair of ring electrodes.

12. The catheter of claim 11, wherein the plurality of control leads further includes:
a separate control lead for each electrode of the plurality of electrodes.

13. The catheter of claim 11, wherein the tip ring electrode is separated axially along the longitudinal axis from the tip electrode.

14. The catheter of claim 11, wherein an outer diameter of the pair of ring electrodes is the same as an outer diameter of the distal portion of the elongated structure.

15. The catheter of claim 11, wherein an outer diameter of the pair of ring electrodes is greater than an outer diameter of the distal portion of the elongated structure.

16. The catheter of claim 11, wherein the pair of ring electrodes comprises a first pair of ring electrodes, and wherein the one or more additional electrodes comprises a second pair of ring electrodes.

17. The catheter of claim 16, wherein a distance between electrodes of the first pair of ring electrodes is less than a distance between adjacent electrodes of the first pair of ring electrodes and the second pair of ring electrodes.
